(19) United States
(12) Patent Application Publication
(10) Pub. No.: US 2003/0148991 A1
(43) Pub. Date: Aug. 7, 2003

(54) Combination of lipoic acid and c1 donors for the treatment of disorders of the central nervous system

(76) Inventors: Wolfgang P Hahnlein, Freinsheim (DE); Klaus Kramer, Landau (DE); Oliver Hasselwander, Landau (DE); Walter E Muller, Worms (DE); Gunther P Eckert, Liederbach a. T. (DE)

Correspondence Address: KEIL & WEINKAUF, 1350 CONNECTICUT AVENUE, N.W., WASHINGTON, DC, 20036, US

(21) Appl. No.: 10/275,579

(22) PCT Filed: May 10, 2001
(86) PCT No.: PCT/EP01/05359

(30) Foreign Application Priority Data
May 10, 2000 (DE) 10022856.9

(51) Int. Cl. A61K 31/525; A61K 31/385; A61K 31/7076

(52) U.S. Cl. 514/47; 514/440; 514/251

(57) ABSTRACT

The present invention relates to the use of lipoic acid and C₁ donors, in particular S-adenosylmethionine and/or 5-methyltetrahydrofolate, for the treatment of central nervous system disorders, to compositions with a corresponding active ingredient combination and to compositions in the form of commercial packs with corresponding combination products or single component products for combined use.

The present invention relates to the use of lipoic acid and C₁ donors for nutritional supplementation, in functional foods and for therapeutic purposes in the treatment of central nervous system disorders, to compositions having a corresponding active ingredient combination, and to compositions in the form of commercial packs with corresponding combination products or single component products for combined use.

Lipoic acid is a coenzyme in the oxidative decarboxylation of α-keto acids and is found in virtually every cell in the body. The antiinflammatory, analgesic and cytoprotective properties of lipoic acid, and its antioxidant effect, make it an interesting active ingredient for pharmacy, cosmetics, food science and adjacent areas (Biothiols in Health and Disease, Herausgeber Packer L. and Cadenas E., Marcel Dekker Inc., New York, Basel, Hongkong). Thus, Stoll et al. reported, in Pharmacology Biochemistry and Behavior, Vol. 46, pp. 799-805 (1993) and in Ann. NY Acad. Sci., Vol. 717, pp. 122-128 (1994), respectively that lipoic acid is able to improve the long-term memory of aged mice and the cognitive abilities of rodents. Han D. et al. postulate, in American Journal of Physiology 273: R 1771-1778 (1997), a lipoic acid-mediated protection against glutamate-induced depletion of intracellular glutathione and use this in an attempt to give a mechanistic explanation of the neuroprotective effects of lipoic acid which are observed in rat ischemia models.

Lipoic acid-containing products are currently listed for the treatment of abnormal sensation associated with diabetic polyneuropathy. Formulations of solid salts of lipoic acid are proposed in U.S. Pat. No. 5,990,152. U.S. Pat. No. 5,994,393 relates to another modification of lipoic acid. Useful lipoic acid analogs are proposed in WO 99/45922. Combinations of lipoic acid and vitamins for producing pharmaceuticals are described in EP 0 572 922 A1. A combination of lipoic acid as biocompatible disulfide with an essential fatty acid and, where appropriate, other essential nutrients is mentioned in WO 99/04782.

C₁ metabolism, i.e. the transfer of activated one-carbon units in various states of oxidation, has fundamental importance for a large number of processes important to life. For example, tetrahydrofolate derivatives serve in a whole series of biosyntheses as donors of C₁ units. Examples which may be mentioned are the syntheses of methionine, thymine and glycine, in which particular derivatives of tetrahydrofolate contribute carbon atoms. Deficits in these processes may be manifested by a large number of pathological states.

Ann important methyl group donor is S-adenosylmethionine (SAM). This substance has multilayer importance as ubiquitous metabolite present in all cells and biological fluids. For example, Bottiglieri discusses, in Exp. Opin. Invest. Drugs (1997) 6 (4): 417-426, the neuropharmacological aspects of SAM and their effects on drug treatment of psychiatric and neurological disorders. A similar review is provided by the article by Bottiglieri et al. in Drugs 48 (2): 137-152 (1994). It was suspected at a very early date that there is an association between folic acid deficiency and neurological disorders, especially affective disorders such as depression, so that importance has frequently been attached to folic acid and folic acid metabolites in the treatment of these disorders (Reynolds & Stramentinoli in Pychological Medicine, 13, 705-710 (1983)).

For example, EP 0 482 493 proposes the use of SAM or else other C₁ donors such as 5-methyltetrahydrofolic acid and 5-formyl-tetrahydrofolic acid for the treatment of neurological impairments in AIDS patients. According to WO 99/37155, mental problems such as depressive states can be treated, and cognitive functions in humans and animals can be supported, with compositions which, besides tyrosine, one or more phospholipids, one or more fatty acids and/or St John's wort, also comprise methylating agents such as SAM, 5-MTHF, folate, betaine or trimethylglycine.

SAM is currently listed for the treatment of degenerative joint disorders with inflammatory activation, although as tosylate bis(sulfate) salt. This is because SAM is extremely unstable, which is why EP 0 074 555 has also proposed particular SAM salts and derivatives with improved stability and bioavailability.

The aforementioned C₁ donors 5-methyltetrahydrofolic acid (5-MTHF) and 5-formyltetrahydrofolic acid (5-FTHF) are also being discussed in connection with neurological disorders. Thus, EP 0 382 019 and EP 0 388 827 describe the use of these substances in pharmaceutical formulations with delayed release of active ingredient for the treatment of depressive disorders and organic mental disorders associated with depression (cf. also Guaraldi et al in Annals of Clinical Psychiatry, Vol. 5, 101-105 (1993) and Passeri et al. in Ageing Clin. Exp. Res. 5, 63-71 (1993)).

A homeopathic preparation described for the stimulation of certain immunological parameters is also based on a complex mixture of vitamins, cofactors, metabolites and enzymes, including tetrahydrofolic acid, lipoic acid and SAM.

CNS disorders currently affect large sections of the population. In particular because of the increase in elderly people and the concomitant increase in the prevalence of age-related disorders of the CNS there is a continual increase in the numbers of patients in this area. Ischemic events, demyelinating processes, depositions, tumors, traumata, infections and causes which can often not be identified in detail lead to neuronal changes and often deficits and to concomitant diverse symptoms with impairment or even loss of important brain functions.

It has now been found that certain combined uses of lipoic acid with C₁ donors make it possible effectively to treat central nervous system disorders and thus represent an ideal nutritional supplementation, confer on foods a beneficial effect on health, and have a high therapeutic value.

The present invention therefore relates to the use of at least one lipoic acid, physiologically acceptable derivatives and/or salts thereof and at least one C₁ donor for the treatment of central nervous system disorders.

The treatment according to the invention represents a combination therapy, i.e. the use of at least one lipoic acid, of a physiologically acceptable derivative or salt thereof—also referred to hereinafter as olipoic acid component for simplicity—and the use of at least one C₁ donor—also referred to hereinafter as C₁ donor component for simplicity—takes place in a situation which is, in particular, therapeutically appropriate, especially in relation to optimal efficacy. Thus, the lipoic acid component and the C₁ donor component can in principle be administered together in one formulation or separately in at least two different formulations. The latter possibility includes both concurrent administration, i.e. taking place at essentially the same times or in direct succession, and sequential administration, i.e taking place at different times. A particular embodiment of sequential administration is achieved by alternating administration of the two components, for example with an early/late daily rhythm.

Thus, the present invention relates to the use both of at least one lipoic acid, of a physiologically acceptable derivative or salt thereof and of C₁ donors for the C₁ donor-assisted treatment or for the lipoic acid-assisted treatment of central nervous system disorders. In this sense, the invention relates to compositions for the treatment of central nervous system disorders which are based on a combination of i) at least one lipoic acid, a physiologically acceptable derivative or salt thereof and ii) at least one C₁ donor and, where appropriate, other active ingredients, it being possible for the active ingredient components, in particular components i) and ii), to be formulated together or separately.

The term “lipoic acid” refers according to the invention to 5-(1,2-dithiolan-3-yl)valeric acid, also called thiooctic acid, of the formula I

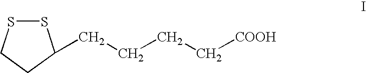

including the optical isomers covered by this formula, both as mixtures, e.g. racemates, and in pure form, e.g. R or S enantiomers. The preferred isomer is (R)-5-(1,2-dithiolan-3-yl)-valeric acid of the formula II

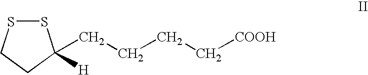

Lipoic acid mixtures with an (R) enantiomeric excess (ee) of at least 40% are preferred. The (R) enantiomeric excess is preferably at least 80%, in particular at least 98%.

Lipoic acid derivatives include, in particular, synthesis precursors and metabolites of lipoic acid, i.e. especially dihydrolipoic acid. Other metabolites which should be mentioned are lipoamide, lipoyllysine, di-6,8-bisnorlipoic acid and tetranorlipoic acid. Other suitable lipoic acid derivatives are, for example, the esters, thioesters and amides of lipoic acid with amino alcohols, amino thiols and diamines which are described in WO 99/45922 as lipoic acid analogs of the formula (I) and which are incorporated in the present application by reference. Corresponding to the statements about lipoic acid, the respective optical isomers of the derivatives also belong therewith.

The physiologically acceptable salts of lipoic acid or lipoic acid derivatives are in the present case preferably base addition salts.

The base addition salts include salts of lipoic acid or lipoic acid derivatives with inorganic bases, for example metal hydroxides or carbonates of alkali metals, alkaline earth metals or transition metals, or with organic bases, for example ammonia or basic amino acids such as arginine and lysine, amines, e.g. methylamine, dimethylamine, trimethylamine, triethylamine, ethylamine, diethylamine, ethylenediamine, ethanolamine, diethanolamine, 1-amino-2-propanol, 3-amino-1-propanol or hexamethylenetetramine, saturated cyclic amines with 4 to 6 ring carbon atoms, such as piperidine, piperazine, pyrrolidine and morpholine, and other organic bases, for example N-methylglucamine, creatine and tromethamine, and quaternary ammonium compounds such as tetramethylammonium and the like.

Salts with inorganic bases are preferred, e.g. Na, K, Mg, Ca, Zn, Cr and Fe salts.

The term “C₁ donor” refers to substances able to transfer one-carbon groups. It is moreover possible for these units to be in different oxidation states, for example as methyl, methylene, formyl, formimino or methenyl group. Methyl groups are preferred. The C₁ donors used in particular according to the invention are those suitable as substrate of C₁ transferases. Mention should be made in this connection in particular of methyltransferases such as homocysteine methyltransferase, also referred to as methionine synthase, betaine-homocysteine methyltransferase and enzymes for methylation of other compounds, e.g. in the conversion of ethanolamine to choline, guanidinoacetate to creatine, N-acetyl-serotonin to N-acetyl-5-methoxyserotonin (melatonin), norepinephrine to epinephrine, certain drugs to methylated drugs, or else of building blocks of particular macromolecules, e.g. nucleic acid bases to methylated bases and histidine residues to 3-methylhistidine residues.

In a further aspect of the present invention, useful C₁ donors are selected from those involved in folate metabolism, in particular N5-methyltetrahydrofolate, N10-formyltetrahydrofolate, N5,N10-methylenetetrahydrofolate, N5-formiminotetrahydrofolate, N5-formyltetrahydrofolate and S-adenosylmethionine.

Further examples of C₁ donors are dimethylglycine and trimethylglycine.

It is particularly preferred according to the invention to use N5-methyltetrahydrofolate (5-MTHF) and/or S-adenosylmethionine (SAM) or physiologically acceptable derivatives and/or salts thereof as C₁ donors.

The term “S-adenosylmethionine” refers according to the invention to 2-amino-3-[(5′-deoxyadenosin-5′-yl)methylsulfonio]butyrate, also called ademetionine, of the formula III

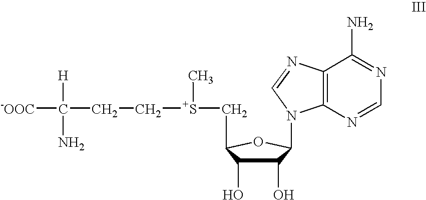

including the optical isomers covered by this formula, both as mixtures, e.g. racemates, and in pure form, e.g. R or S enantiomers. The preferred isomer is (S)-2-amino-3-[(5′-deoxy-adenosin-5′-yl)methylsulfonio]butyrate of the formula IV

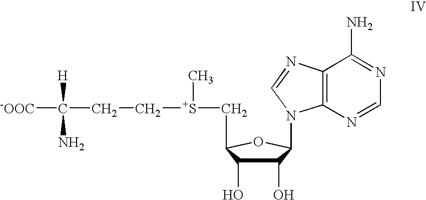

The SAM derivatives include in particular SAM amides and esters. SAM amides and esters which can be hydrolyzed under physiological conditions are advantageous. These derivatives include in particular compounds of the formula V

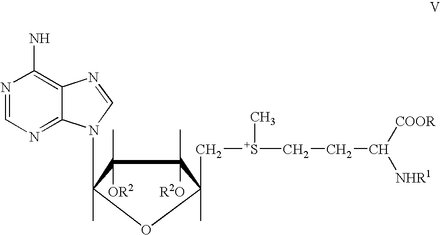

in which R is hydrogen or a linear or branched, saturated or unsaturated, aliphatic radical having 1 to 35 carbon atoms and up to five heteroatoms selected independently of one another from O, N and S, where R is, in particular, radicals derived from natural substances such as vitamins, e.g. tocopherol, cofactors, e.g. lipoic acid, fatty acids, e.g. long-chain aliphatic acids having, preferably, 26 to 32 carbon atoms, amino acids, etc., or is C₁-C₆-alkyl, and R¹ is hydrogen or a linear or branched, saturated or unsaturated, aliphatic or aromatic acyl radical having 2 to 35 carbon atoms and up to five heteroatoms selected independently of one another from O, N and S, where R¹ is, in particular, radicals derived from natural substances such as vitamins, e.g. tocopherol, cofactors, eg. lipoic acid, fatty acids, e.g. long-chain aliphatic acids having, preferably, 26 to 32 carbon atoms, amino acids, etc., is C₂-C₁₂-alkanoyl or is phenyl which is optionally substituted once, twice or three times by C₁-C₆-alkyl, halogen, in partricular fluorine and chlorine, or C₁-C₆-alkoxy, and the R² radicals may be identical or different and have the meanings of R¹. Corresponding to the statements about SAM, the respective optical isomers of the derivatives also belong therewith.

Physiologically acceptable salts of SAM and SAM derivatives include acid and base addition salts and corresponding mixed forms.

The acid addition salts include salts of SAM and SAM derivatives with inorganic acids such as hydrochloric acid, sulfuric acid, nitric acid or phosphoric acid, or organic acids, in particular carboxylic acids, e.g. acetic acid, tartaric acid, lactic acid, citric acid, malic acid, mandelic acid, ascorbic acid, maleic acid, fumaric acid, gluconic acid or sulfonic acids, e.g. methanesulfonic acid, benzenesulfonic acid and toluenesulfonic acid, and the like.

The base addition salts include salts of SAM and SAM derivatives for example with the inorganic or organic bases mentioned above in connection with lipoic acid and lipoic acid derivatives.

For illustration, mention may be made of the SAM derivatives and salts described in EP 0 074 555.

Preferably used according to the invention are the 1,4-butanedisulfonate, the tosylate and/or the sulfate of SAM.

The-term “5-methyltetrahydrofolate” refers according to the invention to 5-methyl-N-[5,6,7,8-tetrahydropteroyl]glutamic acid of the formula VI

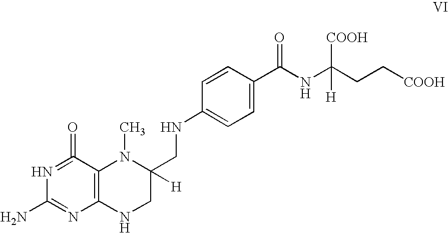

including the optical isomers covered by this formula, both as mixtures, e.g. racemates, and in pure form, eg. R or S enantiomers. Preference is given to the 6(R,S) racemate and the (6S) enantiomer, in particular 5-methyl-N-[(6S)-5,6,7,8-tetra-hydropteroyl]-L-glutamic acid of the formula VII

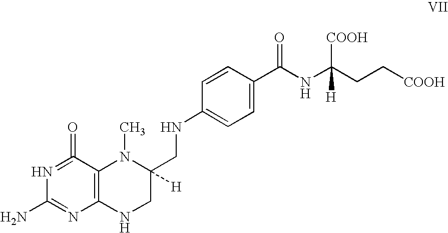

As with the SAM derivatives, the 5-MTHF derivatives include in particular 5-MTHF amides and esters. 5-MTHF amides and esters which can be hydrolyzed under physiological conditions are advantageous. Corresponding to the statements about 5-MTHF, the respective optical isomers of the derivatives also belong therewith.

Physiologically acceptable salts of 5-MTHF and 5-MTHF derivatives include acid and base addition salts and corresponding mixed forms.

The acid addition salts include salts of 5-MTHF and 5-MTHF derivatives for example with the inorganic or organic acids mentioned above in connection with SAM or SAM derivatives.

The base addition salts include salts of 5-MTHF and 5-MTHF derivatives for example with the inorganic or organic bases mentioned above in connection with lipoic acid and lipoic acid derivatives.

It is preferred according to the invention to use the Ca salts of the (6S) enantiomer or of the 6(R,S) racemate.

Besides the lipoic acid and C₁ donor components, the treatment according to the invention may include other active ingredients. These active ingredients may be, in particular, ones whose effect is similar or supplementary to the effect mediated by lipoic acid or C₁ donor. Thus, it may be advantageous to administer in addition to the combination of the invention, antioxidants, antidementia drugs, antiepileptics, geriatrics, antiparkinson agents and agents for other extrapyrimidal disorders, psychotropic drugs and similar active ingredients. Vitamins, cofactors, trace elements and other essential nutrients may also be expedient. Polyunsaturated fatty acids, especially ψ-3- and ψ-6-PUFA, e.g. docosahexaenoic acid and arachidonic acid; neurotransmitters and their precursors, especially dopamine, serotonin, acetylcholine or choline, lecithin, betaine and tyrosine; phospholipids, especially phosphatidylserine and phosphatidylethanolamine; antioxidants, especialy vitamin E and C, flavonoids, tocotrienols etc. are preferably administered together with the lipoic acid component and the C₁ donor component and, in particular, SAM or 5-MTHF.

A particular embodiment of the present invention is based on the combination of lipoic acid, physiologically acceptable lipoic acid derivatives and/or salts thereof with SAM, physiologically acceptable SAM derivatives and/or salts thereof.

Another particular embodiment of the present invention is based on the combination of lipoic acid, physiologically acceptable lipoic acid derivatives and/or salts thereof with 5-MTHF, physiologically acceptable 5-MTHF derivatives and/or salts thereof.

Another particular embodiment of the present invention is based on the combination of lipoic acid, physiologically acceptable lipoic acid derivatives and/or salts thereof with SAM, physiologically acceptable SAM derivatives and/or salts thereof, and with 5-MTHF, physiologically acceptable 5-MTHF derivatives and/or salts thereof.

The invention encompasses within the scope of therapeutic uses a nutritional supplementation, a dietary nutritional strategy or in the area of fortified foods (functional foods) a treatment of individuals.

In the area of nutritional supplementation, the intake ensured by the normal diet is supplemented by an active ingredient combination of the invention. In this sense, the active ingredient combination of the invention is also to be regarded as a nutrient combination. The purpose of this nutritional supplementation may be to compensate for corresponding dietary deficiencies or ensure an intake of these active ingredients which is above the amount ensured with the normal diet. Thus, the use according to the invention for nutritional supplementation also serves physiological dietary purposes, in particular the treatment of corresponding deficiency symptoms and alteration of particular states of an individual, which can be respectively compensated and brought about with a nutritional supplementary intake of the active ingredient combination of the invention. The deficiency symptoms and alterable states include the disorders which can be treated, and effects which can be achieved, according to the invention which are listed below.

The use according to the invention for therapeutic purposes relates in particular to the treatment of central nervous system disorders. By these are meant disorders which affect the spinal cord and, in particular, the brain The term “disorder” in the sense according to the invention refers to abnormalities which are usually regarded as pathological states and may be manifested in the form of certain signs, symptoms and/or dysfunctions. The treatment according to the invention may be directed at individual disorders, i.e. abnormalities or pathological states, but it is also possible for several abnormalities which are, where appropriate, causally connected together to combine to give patterns, i.e. syndromes, which can be treated according to the invention.

The disorders which can be treated according to the invention include in particular neurological and psychiatric disorders.

Neurological disorders include neurodegenerative disorders, in particular neurodegenerative disorders associated with aging processes, demyelinating processes, ischemic events and/or other morphological changes. The morphological changes include, in particular, those associated with neuronal changes and, in particular, deficits, e.g. with infections, traumata, tumors, depositions and/or diffuse atrophic changes in the brain.

Neurological disorders which can be treated according to the invention include impairments of mental functions, especially dementia, in particular cerebrovascular dementia and Alzheimer-type dementia, e.g. senile dementia and Alzheimer's disease, in particular intellectual deficits such as attention deficit disorders, amnesic and cognitive disorders, e.g. learning and memory impairment (impaired cognitive function); multiple sclerosis; parkinsonism; epilepsy; delirium; disorders of attention and waking/sleeping behavior, in particular behavioral disturbances and emotional disturbances starting in childhood and adolescence, such as hyperactivity in children; narcolepsy and sleep disturbances, e.g. restless legs syndrome; developmental disturbances.

Psychiatric disorders include psychoses, e.g. of the acute exogenous type or associated psychoses with an organic or exogenous cause, e.g. after trauma, especially brain lesions and diffuse brain damage, associated with metabolic disorders, infections and endocrinopathies; endogenous psychoses such as schizophrenia and schizotypal and delusional disorders; affective disorders such as depression, mania and manic/depressive states; and combined forms of the disorders described above; neurotic and somatoform disorders, and disorders associated with stress; dissociative disorders, e.g. deficits, clouding and splitting of consciousness and personality disorders; anxiety states; disorders of the sex life, e.g. male impotence; depressive states associated with other disorders, e.g. associated with fibromyalgia and chronic fatigue syndrome; eating disorders, e.g. anorexia or bulimia; and other undefined psychiatric disorders.

Preferred embodiments of the present invention are directed at the treatment of impairments of cognitive functions, in particular of impairments of learning and memory, especially of dementia.

Another preferred embodiment of the present invention is directed at the treatment of depression.

CNS disorders associated with aging are treated in particular.

Disorders to be treated according to the invention are frequently characterized by a progressive development, i.e. the states described above change over the course of time, the severity usually increasing, and, where appropriate, states possibly interchanging or other states being added to previously existing states.

It is possible by the treatment according to the invention to treat a large number of signs, symptoms and/or dysfunctions associated with the disorders and, in particular, the aforementioned states. These include, for example, symptoms of dementia, in particular those affecting social relations, a decline in intellectual functions, e.g. confusion, especially temporal and spatial, disorders of memory and association, and of the capacity for abstract thought and judgement, an impaired relation to reality, lack of insight and the ability to comply with the usual social norms and demands of life, changes in behavior, changes in individual urges such as hunger, sleep, thirst etc., and in mood, personality changes, especially emotional lability, hallucinations, ego disturbances, incoherence of thought, ambivalence, autism, depersonalization or hallucinations, delusional ideas, staccato speech, absence of synkinesis, small-step gait, bent posture of trunk and limbs, tremor, mask-like face, monotonous speech, depression, apathy, deficient spontaneity and irresolution, reduced association ability, anxiety, nervous agitation, stamering, social phobia, panic disorders, expansive syndromes, states of agitation and confusion, dysphoria, dyekinetic syndromes and tic disorders, e.g. associated with Huntington's chorea, Gilles de la Tourette syndrome, vertigo syndromes, e.g peripheral postural, rotational and vestibular vertigo, melancholia, hysteria, hypochondria and the like.

The treatment according to the invention gains importance in adults with increasing age. The treatment has particular advantages in the group of those over 40 years of age and especially in those over 50 years of age.

One aspect of a treatment in the sense according to the invention relates to the treatment of acute or chronic signs, symptoms and/or dysfunctions; one purpose of this treatment is to alleviate the signs, symptoms and/or dysfunctions. A further aspect relates to a preventive treatment (prophylaxis), in particular as recurrence or episode prophylaxis; one purpose of this treatment is to avoid the occurrence of the signs, symptoms and/or dysfunctions, including a postponement of the occurrence. The treatment may be symptomatic, for example directed at suppression of symptoms. It may take place short-term, be directed at the medium term or may also be a long-term treatment, for example as part of maintenance therapy.

The novel use of the described active ingredients comprises a method within the scope of the treatment. This entails the individual to be treated, preferably a mammal, in particular a human or agricultural or domestic animal being given an effective amount of lipoic acid component and an effective amount of C₁ donor component, usually formulated in accordance with the practice of pharmacy, veterinary medicine or food technology. Whether such a treatment is indicated, and the form it is to take, depend on the individual case and may be subject both to specialist medical (usually objective diagnosis) and nonspecialist assessment (usually self-diagnosis) which may take account of the signs, symptoms and/or dysfunctions present, the risks of developing certain signs, symptoms and/or dysfunctions, and other factors.

The treatment usually takes place by single or multiple daily dosage, where appropriate together or alternately with other active ingredients or active ingredient-containing products, so that an individual to be treated is given a daily dose of about 1 mg to 5 g, preferably from about 10 mg to 1 g, of lipoic acid and of about 10 μg to 10 g, preferably from about 100 μg to 2 g, of at least one C₁ donor, for example of about 10 mg to 10 g, preferably from about 25 mg to 2 g, of SAM or of about 10 μg to 20 mg, preferably from about 100 μg to 10 mg, of 5-MTHF, on oral administration, and of about 5 mg to 1 g of lipoic acid, about 10 mg to 1 g of SAM and about 100 μg to 5 mg of 5-MTHF, on parenteral administration.

The quantities and proportions of active ingredients are based on the active ingredient so that an appropriate conversion is necessary for salts and derivatives.

The invention also relates to the production of compositions for the treatment of an individual, preferably a mammal, in particular a human or agricultural or domestic animal.

The present invention therefore also relates in one aspect to compositions comprising

i) at least one lipoic acid, physiologically acceptable lipoic acid derivatives and/or salts thereof, and

ii) at least one C₁ donor and,

where appropriate, at least one other active ingredient and a formulation base.

Compositions of the invention are therefore based on an active ingredient combination and, where appropriate, a formulation base.

The compositions include, in particular, pharmaceutical compositions, nutritional supplements and foods, in particular functional or dietetic foods. The novel foodstuffs and food supplements have, besides a predominantly nutrition-related function, additionally an active ingredient-related function which relates in particular to the active ingredient combination of the invention. They are therefore referred to as functional or dietetic foods or nutrients. Nutritional supplements serve to supplement the daily diet with the active ingredient combination of the invention, with the nutrition-related function of the nutritional supplement on its own becoming of less importance.

The active ingredient combination for the purpose of the invention comprises as active ingredient component i) at least one lipoic acid, a physiologically acceptable derivative or salt thereof. Mixtures of these forms are possible, but will be considered only in certain cases. In a particular embodiment, the active ingredient component i) consists of lipoic acid, preferably at least 90% by weight and, in particular, at least 99% by weight of the (R) enantiomer, where the percent by weight data are based on the total weight of active ingredient component i).

The active ingredient combination additionally comprises for the purposes of the invention as active ingredient component ii) at least one C₁ donor. The active ingredients listed above as particular C₁ donors are preferred, especially SAM, physiologically acceptable SAM derivatives and/or salts thereof, and 5-MTHF, physiologically acceptable 5-MTHF derivatives and/or salts thereof. Mixtures of these forms are possible, but should be considered only in particular cases. In a particular embodiment, the active ingredient component ii) consists of SAM, preferably at lest 90% by weight and in particular at least 99% by weight of the (S) enantiomer. In another particular embodiment, the active ingredient component ii) consists of 5-MTHF, preferably at least 90% and in particular at least 99% by weight of the (6S) enantiomer or the 6(R,S) racemate. In another particular embodiment, the active ingredient component ii) consists of a mixture of SAM and 5-MTHF, with the above statements about preferred compositions applying correspondingly. In these cases too, the percent by weight data are based on the total weight of active ingredient component ii).

The active ingredient combination for the purpose of the invention may additionally comprise as active ingredient component iii) other active ingredients, for example the active ingredients mentioned in this connection above.

The proportion of active ingredient combination in the formulation is larger than the proportion present where appropriate in natural sources, especially foods. In this sense, the novel compositions are enriched in relation to the active ingredient combination. The proportion of active ingredient combination of i) and ii) in the formulation is preferably greater than about 0.01% by weight, advantageously greater than about 0.05% by weight and, in particular, greater than about 0.1% by weight. In the case of a pharmaceutical composition, the proportion is usually about 1 to 60% by weight, preferably about 5 to 35% by weight and, in particular, about 10 to 30% by weight, and in the case of a nutritional supplement and especially in the case of foods where appropriate correspondingly lower if the formulation is given in larger amounts.

Unless otherwise indicated, data in percent by weight are based on the total weight of the formulation.

The formulation base for pharmaceutical formulations of the invention comprises physiologically acceptable excipients. Physiologically acceptable excipients are those known to be usable in the sectors of pharmacy, food technology and adjacent areas, in particular the excipients listed in relevant pharmacopeias (e.g. DAB, Ph. Eur., BP, NF), and other excipients whose properties do not stand in the way of physiological use. Excipients for the purpose of the invention may also have a nutritional value and are therefore generally used as food component. They may also include nutrients, especially essential nutrients.

Suitable excipients may be: wetting agents; emulsifying and suspending agents; preservatives; antioxidants; antiirritants; chelating agents; tablet coating aids; emulsion stabilizers; film formers; gel formers; odor-masking agents; masking flavors; resins; hydrocolloids; solvents; solubilizers; neutralizers; permeation promoters; pigments; quaternary ammonium compounds; refatting and superfatting agents; ointment, cream or oil bases; silicone derivatives; spreading aids; stabilizers; sterilants; suppository bases; tablet excipients such as binders, fillers, lubricants, disintegrants or coatings; propellants; desiccants; opacifiers; thickeners; waxes; plasticizers; white oils. An arrangement concerning this is based on specialist knowledge as described, for example, in Fiedler, H. P., Lexikon der Hilfsetoffe für Pharmazie, Kosmetik und angrenzende Gebiete, 4th edition, Aulendorf: ECV-Editio-Cantor-Verlag, 1996.

Food components usually comprise one or more amino acids, carbohydrates or fats and are suitable for the human and/or animal diet. They comprise individual components, frequently vegetable but also animal products, especially sucrose, where appropriate in the form of syrups, fruit preparations such as fruit juices, nectar, fruit pulps, purses or dried fruit, for example apple juice, grapefruit juice, orange juice, apple purée, tomato sauce, tomato juice, tomato puree; cereal products such as wheat flour, rye flour, oat flour, corn flour, barley flour, spelt flour, corn syrup and starches from said cereals; dairy products such as milk protein, whey, yoghurt, lecithin and lactose.

Essential nutrients include, in particular, vitamins, provitamins, trace elements, amino acids and fatty acids. Essential amino acids which may be mentioned are isoleucine, leucine, lysine, methionine, phenylalanine, threonine, tryptophan and valine. They also include semiessential amino acids which must be given, for example, in periods of growth or deficiency states, such as arginine, histidine, cysteine and tyrosine. Trace elements which may be mentioned are: essential trace elements which have been proved to be necessary for humans and deficiency of which leads to manifestation of clinical symptoms: iron, copper, zinc, chromium, selenium, calcium, magnesium, potassium, lithium, cobalt, molybdenum, iodine, silicon, fluorine, manganese. Likewise elements whose function in humans is as yet inadequately verified: tin, nickel, vanadium, arsenic, manganese. Fatty acids essential for humans which may be mentioned are: linoleic acid and linolenic acid. A comprehensive list of vitamins are to be found in “Referenzwerte für die Nährstoffzufuhr”, 1st edition, Umschau Braus Verlag, Frankfurt am Main, 2000, edited by the Deutsche Gesellschaft für Ernährung.

The total of active ingredient component and formulation base is usually 100% by weight.

Examples of suitable formulations for nutritional supplementation are capsules, tablets, pills, powder sachets, liquid ampuls and bottles with integral droppers, as well as the drug forms mentioned below.

Examples of suitable pharmaceutical formulations are solid drug forms such as oral powders, dusting powders, granules, tablets, especially film-coated tablets, pastilles, sachets, cachets, sugar-coated tablets, capsules such as hard and soft gelatin capsules, suppositories or vaginal drug forms, semisolid drug forms such as ointments, creams, hydrogels, pastes or plasters, and liquid drug forms such as solutions, emulsions, especially oil-in-water emulsions, suspensions, for example lotions, preparations for injection and infusion, eye drops and ear drops. It is also possible to use implanted delivery devices for administering active ingredients of the invention. Liposomes or microspheres may also be used.

Foodstuff formulations usually have the normal form and are preferably marketed in the form of infant food, breakfast products, especially in the form of mueslis or bars, sports beverages, complete meals, especially in the framework of completely balanced diets, dietary products such as diet beverages, diet meals and diet bars.

The formulations are preferably administered by the oral route but they can also, especially in the drugs sector, be administered by the rectal, transdermal, subcutaneous, intravenous, intramuscular or intranasal route.

For producing the compositions, the active ingredients are usually mixed or diluted with a suitable excipient. Excipients may be solid, semisolid or liquid materials serving as vehicle, carrier or medium for the active ingredient. Admixture of other excipients takes place, if necessary, in a manner known per se. It is possible to carry out shaping steps, where appropriate in conjunction with mixing processes, e.g. a granulation, compression and the like.

The active ingredient components can, in particular, be formulated together. However, they may also be initially processed separately and subsequently combined in a compartmented, e.g. multilayer, drug form. It is possible in this way to take account of possible active ingredient incompatibilities and different active ingredient properties such as bioavailability, stability, solubility and the like. Thus, it is preferred to formulate at least active ingredient component i) with delayed release characteristics in a manner known per se. An acid-resistant formulation of SAM is preferred for stability reasons.

The present invention further relates to compositions in the form of a commercial pack having at least one composition based on

i) at least one lipoic acid, physiologically acceptable derivatives and/or salts thereof, and/or

ii) at least one C₁ donor,

where appropriate together with instructions for the therapeutic use of lipoic acid, lipoic acid derivatives or salts thereof in combination with at least one C₁ donor.

One embodiment of this aspect of the invention relates to commercial packs having at least one, in particular pharmaceutical, composition of the type described above with an active ingredient combination of the invention. This embodiment also encompasses commercial packs having a plurality of combination products in diverse dosages or formulations. commercial packs of this embodiment accordingly comprise active ingredient components i) and ii) formulated together.

Another embodiment relates to commercial packs having two or more, in particular pharmaceutical, compositions which are spatially separated from one another and of which at least two compositions comprise different active ingredients. These compositions may be, in particular, single-component products, i.e especially those with active ingredient component i) or ii). In these cases, the commercial pack contains instructions in the sense of the invention for the combined use of the compositions comprising i) and ii). Commercial packs of this embodiment accordingly comprise active ingredient components i) and/or ii) formulated separately, i.e. in the form of at least two spatially separate compositions.

Another embodiment relates to commercial packs having at least one, in particular pharmaceutical, composition based on

i) at least one lipoic acid, physiologically acceptable derivatives or salts thereof; or

ii) at least one C₁ donor.

These take the form of single-component products. In these cases, the commercial pack contains instructions in the sense of the invention for the therapeutic use of the composition in combination with the other active ingredients which form the active ingredient combination of the invention but are not part of the commercial pack, in the form of at least one other composition. Commercial packs of this embodiment accordingly comprise part of the active ingredient combination of the invention. The part which is not contained is included as intended as part of the enclosed instructions.

It is self-evident that commercial packs of the invention may also comprise other products, in particular active ingredient-containing formulations, and comprehensive instructions also going beyond the aforementioned contents.

The present invention is explained in detail by means of the following examples without being restricted thereto.

### EXAMPLE 1

Pharmaceutical Compositions

a) Film-coated tablet with lipoic acid and SAM
(lipoic acid 50 mg + SAM 200 mg)

| | | |
|---|---|---|
| SAM as 1,4-butanedisulfonate | 400 | mg |
| Lipoic acid | 50 | mg |
| Microcrystalline cellulose | 250 | mg |
| Kollidon CL | 25 | mg |
| Colloidal silica | 5 | mg |
| Mg stearate | 4 | mg |
| Coating: Kollicoat MAE 30 DP | 20 | mg |
| Propylene glycol | 3 | mg |
| Talc | 4 | mg |
| b) Film-coated tablet with lipoic acid and 5-MTHF | | |
| (lipoic acid 100 mg + 5-MTHF 5 mg) | | |
| Lipoic acid | 100 | mg |
| 5-MTHF (as Ca salt) | 6.8 | mg |
| Microcrystalline cellulose | 200 | mg |
| Kollidon CL | 10 | mg |
| Colloidal silica | 3 | mg |
| Mg stearate | 3 | mg |
| Coating: | | |
| Pharmacoat 600 | 6 | mg |
| Kollidon VA 64 | 4 | mg |
| Talc | 2 | mg |
| TiO₂ | 2 | mg |
| c) Film-coated tablet with lipoic acid, SAM and 5-MTHF | | |
| (SAM 200 mg + lipoic acid 50 mg + MTHF 1 mg) | | |
| SAM as 1,4-butanedisulfonate | 400 | mg |
| Lipoic acid | 50 | mg |
| 5-MTHF as Ca salt | 1.4 | mg |
| Microcrystalline cellulose | 200 | mg |
| Kollidon CL | 25 | mg |
| Colloidal silica | 6 | mg |
| Mg stearate | 5 | mg |
| Coating: | | |
| Kollicoat MAE 30 DP | 25. | mg |
| Propylene glycol | 4 | mg |
| Talc | 5 | mg |

### EXAMPLE 2

Functional Food

| a) Bar with lipoic acid, SAM and 5-MTHF | | |
|---|---|---|
| (SAM 100 mg + 25 mg lipoic acid + | | |
| 0.5 mg MTHF/bar (60 g)) | | |
| SAM as 1,4-butanedisulfonate | 200 | mg |
| Lipoic acid | 25 | mg |
| 5-MTHF as Ca salt | 0.7 | mg |
| Fructose syrup | 4.2 | g |
| Glucose | 12 | g |
| Carainelized sugar | 3 | g |
| Glycerol | 3 | g |
| Lecithin | 124.3 | mg |
| Hydrogenated vegetable oil | 1.25 | g |
| Roasted oat flakes | 19.6 | g |
| Puffed rice | 7 | g |
| Roasted and chopped almonds | 5.6 | g |
| Coconut flakes | 4 | g |
| b) Muesli with lipoic acid, SAM and 5-MTHF | | |
| (SAM 200 mg + lipoic acid 50 mg + | | |
| 1 mg 5-MTHF/100 g muesli) | | |
| oat flakes | 40 | g |
| Wheat flakes | 27 | g |
| Raisins | 13 | g |
| Dried apple slices | 5.45 | g |
| Dried apricots | 3 | g |
| Wheatgerm | 3 | g |
| Roasted and ground hazelnuts | 6 | g |
| Fortified milk powder comprising | 2.55 | g |
| SAM as 1,4-butanedisulfonate | 400 | mg |
| Lipoic acid | 50 | mg |
| 5-MTHF as Ca salt | 1.4 | mg |

### EXAMPLE 3

Biological Activity of Lipoic Acid and S-adenosylmethionine

The lipid Composition of cell membranes changes with increasing age. In particular there is an increase in the cholesterol content in relation to the phospholipids These changes lead to a decrease in membrane fluidity in neuronal cell membranes. The decrease in fluidity leads inter alia to reduced activity of neurotransmitter receptors and is thus a cause of central nervous system disorders. Aging and central nervous system disorders are additionally accelerated by increased oxidative stress. Oxidative stress is produced by free radicals. These arise through consumption of oxygen by the cells and are crucially involved in the development of central nervous system disorders.

The effect of lipoic acid and SAM on these processes was determined as follows.

A) Measurement of Membrane Fluidity

The fluidity of whole brain membranes (without cerebellum, medella oblongata and brain stem) from young (3 months) and old (22 months) NMRI mice was determined by fluorescence spectroscopy.

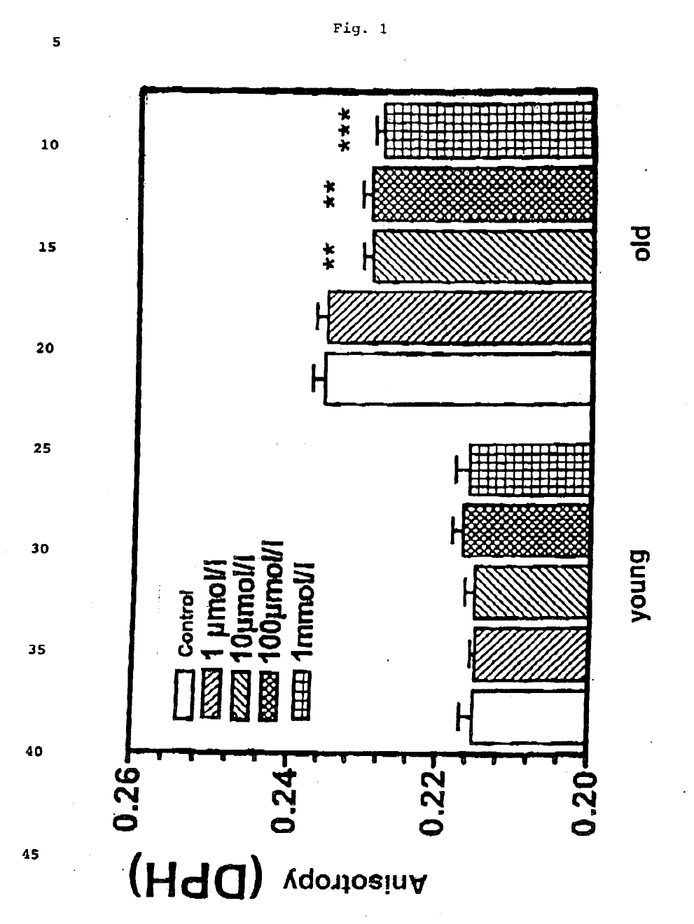

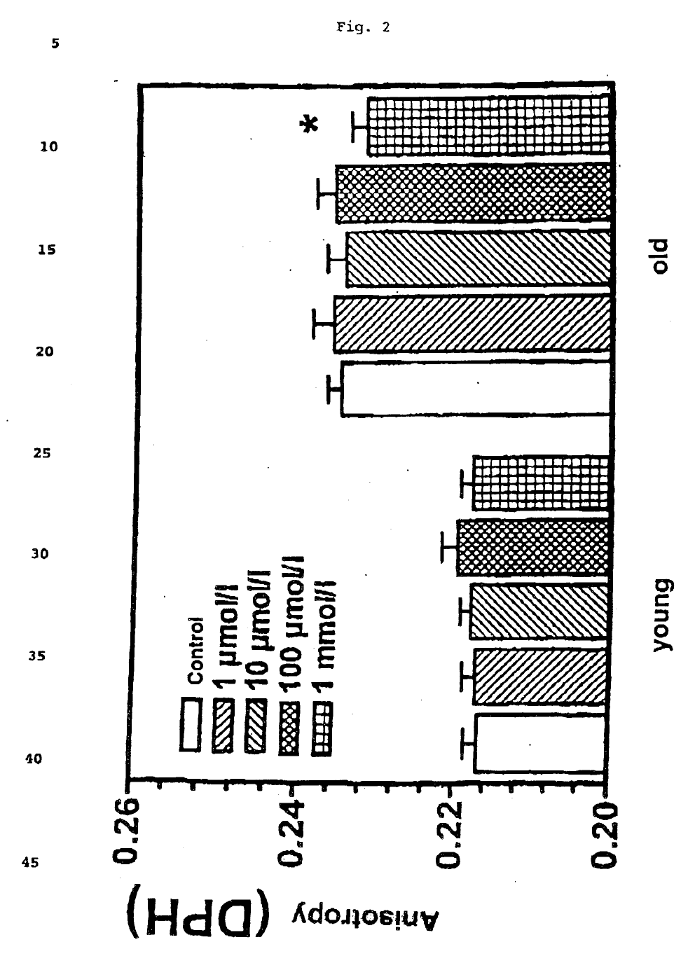

It is known from earlier investigations that brain aging leads to substantial changes in the neuronal membrane structure. Thus, an increase in DPH anisotropy was observed with increasing age. This finding can be confirmed in the present investigations. The DPH anisotropy of whole brain membranes of old mice is increased compared with young mice (FIGS. 1 and 2; control).

Mouse whole brain membranes were prepared by a modification of the method of Cohnen and Müller, 1993. The deep-frozen hemispheres were, after weighing, transferred into 15 ml of ice-cold buffer IV (Tris-HCl buffer 5 mmol/l; pH 7.4), homogenized using a homogenizer (Potter, glass homogenizer with Teflon pestle, from Braun) at about 1 100 rpm by moving the Teflon pestle up and down about ten times, and transferred into centrifuged tubes (PP30 ml, from Schubert). The pestle and the Potter vessel were rinsed with 2×3 ml of ice-cold (4° C.) buffer IV, and the brain homogenate was centrifuged at 48 000 g (=20 000 rpm) and at 4° C. for 20 min (Beckmann J2-21, cooled centrifuge, rotor JA-20). The supernatant was discarded, and the resulting pellet was taken up in 20 ml of ice-cold buffer IV. The pellet was resuspended using an Ultra-Turrax and was centrifuged again under the same conditions. The pellet resulting therefrom was deep-frozen at −20° C.

Membrane fluidity can be measured inter alia by fluorescence spectroscopic methods. In fluorescence polarization spectroscopy, a membrane-bound fluorescent dye is brought to an excited energy state by polarized light of a particular wavelength. The metastable excitation state exists for about 10⁻⁹ to 10⁻⁸ seconds, and then the molecule returns to the energy groundstate with discrete light emission. The polarization of the emitted light varies depending on the mobility of the fluorescent dye in the membrane. Although the mobility of the fluorescent probe incorporated into the lipid bilayer is restricted within the membrane, it can move parallel and perpendicular to the surface to a certain extent. This mobility in certain directions is referred to as anisotropic. Membrane fluidity is therefore also indicated as fluorescence anisotropy besides the fluorescence polarization. The anisotropy is in this case calculated from the measured polarization.

The fluorescent dye diphenylhexatriene (DPH) was used for this purpose. After incorporation of this dye it is located axial with respect to the fatty acid residues of the membrane phospholipids. The linear fluorophores thus represent the flexibility of the fatty acid residues. The dye varies the polarization of the emitted light depending on the rotational mobility in the membrane. This process is measured as anisotropy.

An SLM Aminco 4800 polarization spectrometer (from SLM Aminco) or an SLM Bowman II polarization spectrometer (from SLM Aminco) was employed for the fluidity measurements. The detectors are arranged in the T format in the former apparatus and in the L format in the latter. The T format apparatus is located at the Zentralinstitut für Seelische Gesundheit in Mannheim. When the institute was moved to Frankfurt, the experiments were continued with the newly acquired L format apparatus. Comparative measurements showed that the two apparatuses provided equivalent results.

The light source in both apparatuses was a xenon are lamp (from Osram, Munich) with a 270-700 nm light spectrum. The attached monochromator generated a light beam with an excitation wavelength of 360 nm, which [lacuna] the excitation maxima of DPH.

An emission monochromator inserted between the polarizer and PMT was used for detection at the optimal emission wavelength of 450 nm. Quartz cuvettes (QS-101, from Helma) were used for measurement, being suitable for fluorescence spectroscopy because of their transparency for ultraviolet light. An external thermostat ensured a constant temperature of 37° C. in the sample chamber. Depolarization of the emitted fluorescent light is eventually determined by measuring the ratio of the light intensities passing through the vertically oriented (I//) and the horiziontally oriented polarizer (I⊥). The DPH anisotropy of the whole brain membrane was measured without and with incubation of SAM or lipoic acid (concentrations: 1, 10, 100 μmol/l and 1 mmol/l).

B) Measurement of the Production of Free Radicals

Oxidative stress or the production of free radicals was measured using the fluorescence dye dihydrorhodamine (DHR 123). Brain cells were enriched by incubation with DHR 123 in a waterbath at 37° C. for 15 minutes. DHR is then located in particualr in the mitochondria and fluoresces after oxidation by free radicals. Fluorescence was measured at an excitation wavelength of 500 nm and an emission wavelength of 536 nm. The generation of free radicals was then measured in the absence (control) and presence of lipoic acid at various concentrations (100 and 500 μmol/l).

The results are summarized in the following figures, which show

FIG. 1 comparison of the anisotropy measured using DPH on exposure of whole brain membranes from young and old mice to the stated SAM concentrations;

FIG. 2 comparison of the anisotropy measured using DPH on exposure of whole brain membranes from young and old mice to the stated R-lipoic acid concentrations;

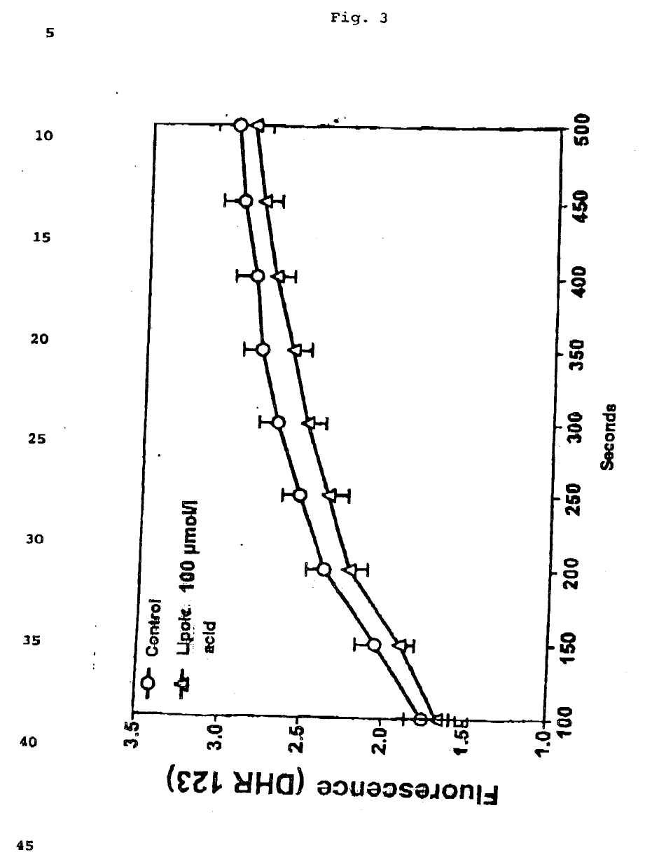

FIG. 3 the time-dependent production of free radicals measured using DHR 123 in brain cells on exposure to the stated R-lipoic acid concentration, compared with the control;

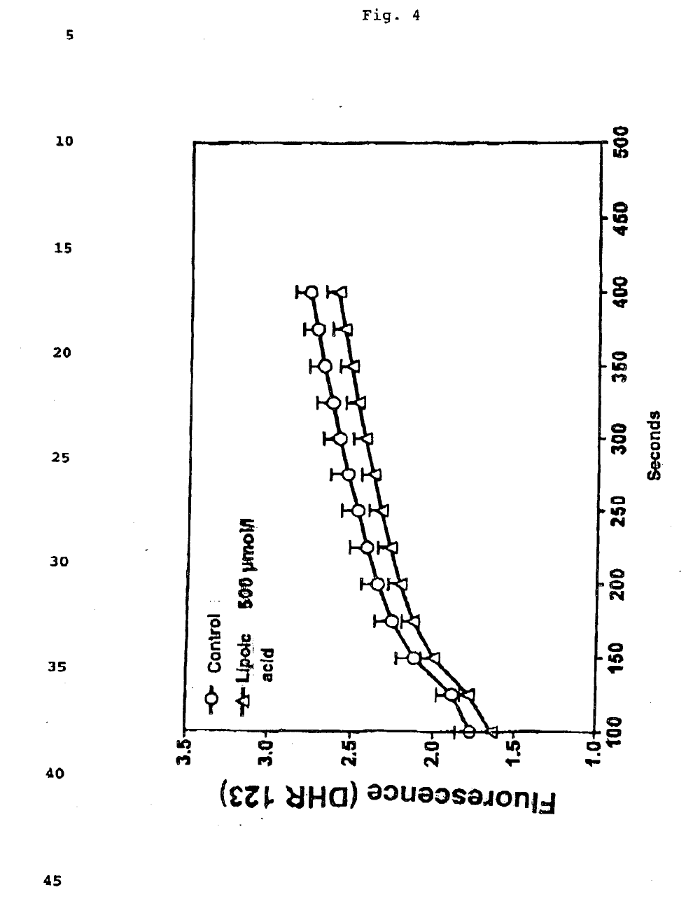

FIG. 4 the time-dependent production of free radicals measured using DHR 123 in brain cells on exposure to the stated R-lipoic acid concentration, compared with the control.

Both SAM and lipoic acid reduce the anisotropy in membranes from old mice (FIGS. 1 and 2). This means that both substances increase membrane fluidity and are thus suitable for the treatment of central nervous system disorders.

Lipoic acid counteracts the generation of free radicals (FIGS. 3 and 4) which was shown here by inhibition of the increase in fluorescence. The generation of free radicals is likewise associated with central nervous system disorders because the radicals are responsible for oxidative damage in the brain.

It is possible to treat central nervous system disorders more effectively by combining SAM and lipoic acid than with SAM or lipoic acid alone. The increase in membrane fluidity in conjunction with the reduced production of free radicals by the active ingredient combination of the invention leads to a synergism in the treatment of certain disorders and, in particular, the disorders which are preferably and advantageously to be treated here.

We claim:

1. The use of at least one lipoic acid, of a physiologically acceptable derivative and/or salt thereof and at least one C₁ donor for producing a composition for C₁ donor-assisted treatment of central nervous system disorders.

2. The use as claimed in claim 1, where the C₁ donor is suitable as substrate of a methyltransferase.

3. The use as claimed in either of the preceding claims, where the C₁ donor is involved in folate metabolism.

4. The use as claimed in any one of the preceding claims, where the C₁ donor is selected from S-adenosylmethionine, 5-methyltetrahydrofolate, physiologically acceptable derivatives and/or salts thereof.

5. The use as claimed in any one of the preceding claims for the treatment of impairments of cognitive functions and of depression.

6. The use as claimed in any one of the preceding claims for the treatment of central nervous system disorders associated with aging.

7. A composition comprising
i) at least one lipoic acid, a physiologically acceptable lipoic acid derivative and/or salt thereof, and
ii) at least one C₁ donor and,
where appropriate, at least one other active ingredient and a formulation base.

8. A composition as claimed in claim 7, wherein the active ingredient content is at least 0.01% by weight.

9. A composition in the form of a commercial pack having at least one composition based on
i) at least one lipoic acid, a physiologically acceptable lipoic acid derivative and/or salt thereof, and/or
ii) at least one C₁ donor,
where appropriate with instructions for the therapeutic use of lipoic acid, lipoic acid derivatives or salts thereof in combination with at least one C₁ donor.
